(19) United States
(12) Patent Application Publication
(10) Pub. No.: US 2019/0071477 A1
(43) Pub. Date: Mar. 7, 2019

(54) Recombinant complement Factor H-immunoglobulin fusion protein with complement regulatory activity, and preparation method therefor and use thereof

(71) Applicant: QUASSIA BIOPHARMA CO., LTD, Changzhou, Jiangsu (CN)

(72) Inventors: Jian-Xin Bao, San Mateo, CA (US); Ya-Ping Lou, San Mateo, CA (US)

(21) Appl. No.: 16/021,034

(22) PCT Filed: Dec. 28, 2016
(86) PCT No.: PCT/CN2016/112504

§ 371 Date: Oct. 12, 2018

(30) Foreign Application Priority Data
Dec. 31, 2015 (CN) 201511028031.9

(51) Int. Cl. C07K 14/47; C12N 15/62; A61K 38/36; A61P 27/02; A61P 37/02; A61P 7/02; A61P 13/12; A61P 31/04; C07K 16/28

(52) U.S. Cl. CPC C07K 14/472; C12N 15/62; A61K 38/36; A61P 27/02; C07K 16/283; A61P 7/02; A61P 13/12; A61P 31/04; A61P 37/02

(57) ABSTRACT

The invention discloses a recombinant complement factor H (CFH)-immunoglobulin (Ig) fusion protein CFH-Ig with complement regulating activity, more specifically, complement regulating activity in the alternative complement pathway, and at the same time with the effect of targeting to tissues or cells where there is overactivation of complement. The invention further relates to a method for preparation of the fusion protein. The invention also relates to a pharmaceutical composition that contains the aforementioned fusion protein for treating autoimmune diseases or other diseases mediated by, or caused by disregulation or deficiency in the alternative complement pathway, as well as preventing or treating thrombosis caused by excessive complement activation in humans or other mammals.

### FIELD OF THE INVENTION

The invention relates to a fusion protein in the field of genetic engineering, and in particular to a recombinant complement Factor H (CFH) fusion protein having a complement regulatory activity, more specifically, to a complement regulatory activity in the alternative complement pathway. The recombinant complement Factor H (CFH) fusion protein consists of a complement Factor H part selected from the group consisting of a full-length CFH, a biologically active CFH fragment, and a combination of fragments thereof, and an immunoglobulin part having an immunoglobulin heavy chain constant region (CH). The invention also relates to a method for preparation and use of the recombinant complement Factor H (CFH) fusion protein having a complement regulatory activity.

### DESCRIPTION OF PRIOR ART

Complement is a general term for a group of immunomodulatory proteins present in serum and tissue fluids of normal humans and animals. It is composed of C1, C2, . . . , and C9 etc, and is a major effector of the innate immune system. Complement is a multi-molecular system consisting of more than 30 soluble proteins, membrane-bound proteins and complement receptors, and therefore it is called the complement system. Based on their biological functions, the components of the complement system can be categorized into the intrinsic complement components, complement regulatory components and complement receptors. The complement system is mainly involved in the targeting and clearance of exogenous pathogens, and also involved in the elimination of immune complexes and cell debris, and enhances cellular immunity. The complement system has also been shown to play an important role in the pathological processes of various autoimmune and inflammatory diseases.

The process of complement activation includes the classical pathway, the Mannose-binding lectin pathway, and the alternative pathway. The classical pathway is triggered by the activation of C1 complex, which consists of one C1q molecule, two C1r molecules, and two C1s molecules. The activation of the classical pathway is initiated when C1q binds to the classical pathway activation factors (mainly antigen-antibody complexes containing IgM, IgG1, IgG2, or IgG3). C1q binds to a single IgM molecule or two adjacent IgG molecules, which in turn activates C1r and C1s. The sequence activation in the classical complement activation pathway is C1, C4, C2, C3, C5, C6, C7, C8 and C9. In the lectin pathway, mannose-binding lectin plays a role in complement activation similar to C1q protein in the classical pathway, binding to mannose residues and fructose residues on the surface of pathogens, and also to MBL (mannose-binding lectin)-associated serine proteases MASP-1 and MASP-2 (similar to C1r and C1s) to form complex to activate complement, leading to complement activation similar to the classical pathway. The activation of the alternative pathway relies on the spontaneous hydrolysis of serum C3 to C3a and C3b, which in turn attaches to the surface of the target cell and binds to Factors B, D, and P, and then proceeds to activation steps analogous to the classical pathway. All three pathways progress very similarly after C3b production. The formed protease C5 convertase breaks C5 into C5a and C5b, which in turn forms a so-called membrane attack complex (MAC) together with C6, C7, C8, and polymeric C9, assembling into transmembrane channel of about 10 nm on the cell membrane of the target cell, leading to target cell swelling and rupture due to its inability to maintain the osmotic pressure.

Under normal circumstances, activation of the complement system occurs only on the surface of invading pathogens without damaging the normal human cells themselves. Complement H (CFH) is a key molecule involved in this “self” and “non-self” recognition during the activation of the alternative complement system. In the alternative pathway, C3b attached to the surface of the target cell binds to complement Factor B, followed by cleavage of the B factor by complement Factor D to produce C3bBb, and C3bBb binds to the Factor P to form C3bBbP (C3 convertase of the alternative pathway). C3bBbP cleaves C3 to produce more C3b, which further causes complement activation, forming a rapidly amplifying positive feedback loop. It is known that CFH binds to C3b and competes for the binding site of Factor B, thereby blocking the formation of C3bBb. CFH also serves as the cofactor for Factor I to degrade C3b to form iC3b, but iC3b cannot bind with Factor B. Moreover, CFH can accelerate the irreversible decay of the already formed C3bBb complex. Therefore, CFH exerts its inhibitory effects on the activation of the alternative pathway through different mechanisms.

CFH is a glycoprotein with a high concentration in the plasma (˜500 μg/mL) and is mainly produced by the liver. Mature CFH consists of 1213 amino acids and is made up with 20 short consensus repeats (SCR) or complement control protein (CCP) modules to form a bead-like structure. These SCRs are termed SCR 1 to SCR 20 in order from the N terminus to the C terminus. Each SCR consists of about 60 amino acids and is highly similar in spatial structure. CFH interacts with C3b mainly through two main binding sites, which are located at SCR (1-4) (binding to intact C3b) and SCR (19-20) (binding to C3d), and it has been reported that SCR (6-14) also binds to C3b (binding to C3c fragments) (Sharma A K & Pangburn M K. Identification of three physically and functionally distinct binding sites for C3b in human complement Factor H by deletion mutagenesis. Proc. Natl. Acad. Sci. USA 1996, 93:10996-11001. Jokiranta T S, et al. Each of the three binding sites on complement Factor H interacts with a distinct site on C3b. J Biol Chem. 2000, 275(36):27657-62.). CFH binds to C3b immobilized on the cell surface and adheres to the cell surface, inhibiting the formation of C3bBb. Existing studies have shown that the complement inhibitory activity domain of CFH is located in SCR (1-4); SCR (1-4) has the ability to bind to C3b, act as a cofactor for complement Factor I, and accelerates C3bBb decay. CFH also has sites for binding to cell surface C3 receptors CR3 and glycosaminoglycans (GAGs), mainly located in SCR7 and SCR (19-20), respectively (Aslam M & Perkins S J. Foldered-back solution structure of monomeric Factor H of human complement by synchrotron X-ray and neutron scattering, analytical ultracentrifugation and constrained molecular modelling. J Mol Biol. 2001, 309(25):1117-1138.). Since GAGs usually exist only on the cell surface of normal animals, and bacteria, viruses and other pathogens do not have such a structure on the surface, CFH protects the self-cells from the membrane attack complex (MAC)-mediated damage caused by excessive activation of the alternative pathway through such specific recognition. SCR (6-8) and SCR (18-20) can also bind to C-reactive protein (CRP) immobilized on the surface of tissues or cells, and reduces tissue damage caused by over-activation of complement system during inflammation (Perkins S J, et al. Complement Factor H-ligand interactions: Self-association, multivalency and dissociation constants. Immunobiology 2012, 217:281-297.).

Aberrant activation of the alternative complement pathway or CFH gene abnormalities such as single nucleotide polymorphisms (SNPs) have been shown to be involved in the pathogenesis of multiple autoimmune diseases and inflammatory reactions. Those involving direct CFH abnormalities include age-related macular degeneration (AMD, Y402H single nucleotide polymorphism in CFH SCR7), ischemic stroke, atypical hemolytic uremic syndrome (aHUS), and schizophrenia and so on. Other pathological processes involving the alternative pathway of complement activation also include local ischemia and reperfusion injury, lupus nephritis, type II membrane proliferative glomerulonephritis (MPGN-II) or dense deposit disease (DDD), rheumatoid arthritis, paroxysmal nocturnal hemoglobinuria (PNH), etc.

It has been shown to be effective for the treatment of certain diseases by targeted inhibition or down regulation of overactivated alternative complement pathway. For example, several patents describe components containing CFH for the treatment of various diseases including eye diseases such as AMD, hemolytic uremic syndrome, autoimmune diseases such as systemic lupus erythematosus, rheumatoid arthritis, and glomerulonephritis and so on. The CR2-CFH fusion protein disclosed in U.S. Pat. No. 7,759,304B2 issued on Jul. 20, 2010 to G. Gilkeson, et al. has a significant alleviating effect in both wet and dry AMD animal models. Additionally, anti-Factor B antibodies, anti-Factor D antibodies, anti-Factor Bb antibodies, and CRIg fusion proteins, etc. are also shown to be effective for the treatment of related diseases.

Most of the above studies selected targets in the complement system that differ from CFH, such as Factor B, Factor D, and Factor Bb, but CFH is considered the most important regulator of the alternative pathway of complement activation, with multiple complement regulatory functions, and has a regulatory role in both the liquid phase (such as blood) and the solid phase (such as cell surface). Although some patent is on CFH, either full-length CFH or different CFH fragment (such as SCR (1-5) in U.S. Pat. No. 7,759,304B2, they mostly describe only single biological activity or effect.

### SUMMARY OF THE INVENTION

It is the aim of the present invention to provide a recombinant complement Factor H (CFH)-immunoglobulin (Ig) fusion protein, having complement regulatory activity, in particular a complement regulatory activity in the alternative complement pathway, and prolonging its half life in vivo.

The invention discloses a recombinant complement factor H (CFH)-immunoglobulin (Ig) fusion protein CFH-Ig with complement regulating activity, especially complement regulating activity in the alternative complement pathway, and at the same time with the effect of targeting to tissues or cells where there is overactivation of complement. The invention further relates to a preparation method of the fusion protein and also to a pharmaceutical composition that contains the aforementioned fusion protein for treating autoimmune diseases or other diseases mediated by, or caused by disregulation or deficiency in the alternative complement pathway, as well as preventing or treating thrombosis caused by excessive complement activation in humans or other mammals.

The following further describes the present invention in detail with reference to specific embodiments.

### DESCRIPTION OF THE DRAWINGS

FIG. 1 is a schematic representation of the expression vector for a recombinant human complement factor CFH-Ig fusion protein and its mature protein, and a modified recombinant human complement factor CFH-Ig fusion protein. A. Expression vector for a recombinant human complement factor CFH-Ig fusion protein, B. The structure of mature recombinant human complement factor CFH-Ig fusion protein (“bivalent”), C. The structure of mature recombinant human complement factor CFH-Ig fusion protein (“multivalent”), D. Modified recombinant human complement factor CFH-Ig fusion protein.

FIG. 2, Panel A shows the results of 1% agarose gel electrophoresis assay of human Xho I-CFH signal peptide-hSCR (1-7)-hFc-6×His-Xba I gene sequence; Panel B shows the results of 1% agarose gel electrophoresis assay of the human Xho I-CFH signal peptide-hSCR (1-7)-mFc-6×His-Xba I gene sequence.

FIG. 3, Panel A shows the results of 1% agarose gel electrophoresis assay for the positive clones of the human Xho I-CFH signal peptide-hSCR (1-7)-hFc-6×His-Xba I expression vector; Panel B shows the results of 1% agarose gel electrophoresis assay for the positive clones of the human Xho I-CFH signal peptide-hSCR (1-7)-mFc-6×His-Xba I expression vector.

FIG. 4 shows the electrophoretograms of the sample of hSCR (1-7)-hFc fusion protein after purification (Panel A) and of the sample of the hSCR (1-7)-mFc fusion protein after purification (Panel B).

FIG. 5 shows Western blot images of hSCR (1-7)-hFc fusion protein (Panel A, lane 1) and hSCR (1-7)-mFc fusion protein (Panel B, lane 1).

FIG. 6 shows the results of 1% agarose electrophoresis assay of the human Xho I-CFH signal peptide-hSCR (1-7)-hSCR (18-20)-hFc-6×His-XbaI gene sequence (lane 1).

FIG. 7 shows the result of 1% agarose electrophoresis detection assay for the positive clones of the human Xho I-CFH signal peptide-hSCR (1-7)-hSCR (18-20)-hFc-6×His-Xba I expression vector.

FIG. 8 is an electropherogram of a sample after purification of the hSCR (1-7)-hSCR (18-20)-hFc fusion protein.

FIG. 9 is a Western blot image of the hSCR (1-7)-hSCR (18-20)-hFc fusion protein.

FIG. 10 shows the comparison of binding affinity for C3b between hSCR (1-7)-hFc, hSCR (1-7)-mFc, hSCR (1-7)-hSCR (18-20)-hFc, and human CFH, where QBC7007 refers to hSCR (1-7)-hFc, QBC7004 refers to hSCR (1-7)-mFc, and QBC7008 refers to hSCR (1-7)-hSCR (18-20)-hFc.

FIG. 11 shows the inhibitory activities on rabbit red blood cell hemolysis mediated by the activation of alternative complement pathway of hSCR (1-7)-hFc, hSCR (1-7)-mFc, hSCR (1-7)-hSCR (18-20)-hFc and human CFH, where QBC7007 refers to hSCR (1-7)-hFc, QBC7004 refers to hSCR (1-7)-mFc, and QBC7008 refers to hSCR (1-7)-hSCR (18-20)-hFc.

FIG. 12 shows the cofactor activity of hSCR (1-7)-hFc, hSCR (1-7)-mFc, hSCR (1-7)-hSCR (18-20)-hFc and human CFH for complement Factor I to cleave C3b. A. Electrophoresis results of cleaved samples, B. Comparison of cleaving rates of different fusion proteins.

### DETAILED DESCRIPTION OF THE INVENTION

Disclosed in the present invention is a recombinant complement Factor H (CFH)-immunoglobulin (Ig) fusion protein, abbreviated as CFH-Ig, having a complement regulatory activity, and consists of:
a) a complement Factor H part selected from the group consisting of a full-length CFH, a biologically active CFH fragment, and a combination of fragments thereof, and
b) an immunoglobulin part having an immunoglobulin heavy chain constant region (CH),
wherein the CFH part has a complement regulatory activity having an effect selected from the group consisting of inhibiting the excessive complement activation, regulating the excessive complement activation, and simultaneously with the aforementioned effects targeting to tissues where there is excessive complement activation; and wherein the complement regulatory activity of the CFH part is an activity in the alternative complement pathway;
wherein the immunoglobulin part has an effect of prolonging its half-life in vivo.

The CFH-Ig fusion protein of the present invention was designed and invented based on the known function of C3b and the known three-dimensional structure of CFH and the model of its interaction with C3b and other ligands. The CFH-Ig fusion protein contains CFH fragment that acts as a cofactor of complement Factor I and accelerates the decay of C3bBb, or simultaneously have a fragment that binds efficiently to C3b and binds to glycosaminoglycans (GAGs) and CRP on cell or particle surface, or a combination of fragments thereof. It is known that C3b produced spontaneously by the alternative pathway is an important opsonin and is one of the components in the three out of the four convertases of the complement system. For example, the formed C3bBb complex is a type of C3 convertase, which cleaves C3 to produce more C3b, and promotes the cascade of activation of complement; the thus formed C3bBbC3b complex is a C5 convertase, which cleaves C5 to produce C5b that eventually participates in the formation of a membrane attack complex (MAC). Excess activation of the alternative complement pathway can cause damage to normal tissues leading to tissue inflammation or cell death. Therefore, effective regulation of C3b levels or its activity can reduce, prevent or even reverse the tissue damage caused by overactivation of the alternative complement pathway. In one embodiment, the CFH portion of the CFH-Ig fusion protein of the present invention contains a CFH fragment that effectively binds C3b, and also contains a CFH fragment that acts as a cofactor of complement Factor I and accelerates the C3bBb decay; in another embodiment, the CFH-Ig fusion protein in the present invention also has CFH fragments that bind to glycosaminoglycans (GAGs) and C-reactive protein (CRP) on tissue, cells or particle surface, or a combination of fragments thereof, to effectively inhibit or regulate complement overactivation of the alternative complement pathway, or at the same time has the effect of targeting tissue where overactivation of complement occurs.

In the present invention, “a biologically active CFH fragment” or “CFH fragment having biological activity” refers to a CFH fragment having a complement regulatory activity, in particular a complement regulatory activity in the alternative complement pathway. Specifically, a biologically active CFH fragment has one or more of the following activities: binding activity to cell surface complement receptor (CR3, CD11b/CD18 or integrin α_{M}/integrin β₂), binding activity to C3b, binding activity to GAGs, and binding activity to CRP, binding activity to pathogens, cofactor activity for complement Factor I cleavage of C3b and C3bBb decay accelerating activity.

In the CFH-Ig fusion protein of the present invention, the recombinant complement Factor H (CFH) is selected from the group consisting of at least one full-length CFH sequence, at least one biologically active N-terminal short consensus repeat (SCR) fragment of CFH from SCR1-SCR17, a combination of at least two different aforementioned SCR fragments each with at least one aforementioned SCR fragment, a combination of at least one aforementioned SCR fragment with C-terminal SCR (18-20) of CFH, a combination of at least one aforementioned SCR fragment with C-terminal SCR (19-20) of CFH, a combination of two different combined aforementioned SCR fragments with C-terminal SCR (18-20) of CFH, and a combination of two different combined aforementioned SCR fragments with C-terminal SCR (19-20) of CFH, wherein the N-terminal SCR fragment of CFH is selected from the group consisting of SCR (1-3), SCR (1-4), SCR (1-5), SCR (1-6), SCR (1-7), SCR (1-8), SCR (1-9), SCR (1-10), SCR (1-11), SCR (1-12)), SCR (1-13), SCR (1-14), SCR (1-15), SCR (1-16) and SCR (1-17).

Preferably the recombinant complement Factor H (CFH) part of the CFH-Ig fusion protein of the present invention is selected from the group consisting of full-length CFH sequence, CFH N-terminal fragment SCR (1-4), CFH N-terminal fragment SCR (1-7), a combination of SCR (1-4) and SCR (18-20), a combination of SCR (1-4) and SCR (19-20), a combination of SCR (1-7) and SCR (18-20), and a combination of SCR (1-7) and SCR (19-20).

More preferably, the recombinant complement Factor H (CFH) part of the CFH-Ig fusion protein of the present invention is selected from the group consisting of CFH fragment SCR (1-7), and a combination of the fragment SCR (1-7) and the fragment SCR (18-20).

The amino acid sequences of human full-length CFH, human CFH fragment SCR (1-4), human CFH fragment SCR (1-7), human CFH fragment SCR (18-20) and human CFH fragment SCR (19-20) in the CFH-Ig fusion protein of the present invention are shown in the Sequence Listing, respectively, as SEQ ID NO:1, SEQ ID NO:2, SEQ ID NO:3, SEQ ID NO:4, and SEQ ID NO:5, or amino acid sequences that have at least 90% homology with SEQ ID NO:1, SEQ ID NO:2, SEQ ID NO:3, SEQ ID NO:4 and SEQ ID NO:5, respectively.

In addition, in the CFH-Ig fusion protein of the present invention, the recombinant complement Factor H (CFH) portion is also selected from the group consisting of two or more CFH full-length sequences, two or more biologically active N-terminal SCR fragments of CFH from SCR1-SCR17, a combination of two or more different aforementioned SCR fragments each with at least one aforementioned SCR fragment, a combination of two or more aforementioned SCR fragment with C-terminal SCR (18-20) of CFH, a combination of two or more aforementioned SCR fragment with C-terminal SCR (19-20) of CFH, a combination of two or more different combined aforementioned SCR fragments with C-terminal SCR (18-20) of CFH, and a combination of two or more different combined aforementioned SCR fragments with C-terminal SCR (19-20) of CFH, wherein the N-terminal SCR fragment of CFH is selected from the group consisting of SCR (1-3), SCR (1-4), SCR (1-5), SCR (1-6), SCR (1-7), SCR (1-8), SCR (1-9), SCR (1-10), SCR (1-11), SCR (1-12)), SCR (1-13), SCR (1-14), SCR (1-15), SCR (1-16) and SCR (1-17).

Preferably, the recombinant complement Factor H (CFH) portion of the CFH-Ig fusion protein of the present invention is selected from the group consisting of 2-4 full-length CFH sequence, 2-4 biologically active N-terminal short consensus repeat (SCR) fragment of CFH from SCR1-SCR17, a combination of 2-4 aforementioned SCR fragment with C-terminal SCR (18-20) of CFH, a combination of 2-4 aforementioned SCR fragment with C-terminal SCR (19-20) of CFH, wherein the N-terminal SCR fragment of CFH is selected from the group consisting of SCR (1-3), SCR (1-4), SCR (1-5), SCR (1-6), SCR (1-7), SCR (1-8), SCR (1-9), SCR (1-10), SCR (1-11), SCR (1-12)), SCR (1-13), SCR (1-14), SCR (1-15), SCR (1-16) and SCR (1-17).

The recombinant complement Factor H (CFH) portion of the CFH-Ig fusion protein of the present invention is derived from humans. In order to verify the pharmacological effects of the CFH-Ig fusion protein of the present invention in other species, the CFH portion may also be derived from the group consisting of mice, rats, guinea pigs, rabbits, dogs, pigs, sheep and non-human primates. Preferably, the CFH portion is derived from the group consisting of humans, mice, rats and non-human primates. More preferably, the CFH portion is derived from humans.

The immunoglobulin (Ig) portion of the CFH-Ig fusion protein of the present invention is derived from the group consisting of humans, rats and mice, preferably from humans. The immunoglobulin (Ig) portion contains an immunoglobulin constant region, immunoglobulin constant region being an immunoglobulin heavy chain constant region (CH), and the immunoglobulin heavy chain constant region is selected from different immunoglobulins such as IgA, IgD, IgE, IgG and IgM, preferably, the immunoglobulin heavy chain constant region is selected from IgG; the immunoglobulin G (IgG) is selected from different subtypes of IgG, IgG1, IgG2 (IgG2a, IgG2b), IgG3 and IgG4, and combinations between different subtypes (e.g., IgG2/IgG4); more preferably, the immunoglobulin heavy chain constant region is derived from IgG1, IgG2, or IgG4.

In order to reduce or prevent effector functions of the immunoglobulin Fc domain, such as activation of complement and/or binding to the antibody receptor (Fc receptor), the amino acids in the Fc domain of IgG1 for the Fc receptor binding site can be deleted or replaced, or it is used IgG4 that does not activate complement or IgG2 or a combination of IgG2 and IgG4 that does not bind to Fc receptors.

The IgG heavy chain constant region may include a CH1 region, a hinge region, a CH2 region, and a CH3 region, including at least a Fc fragment (hinge region, CH2 region, and CH3 region). The Fc portion is an immunoglobulin Fc domain derived from human or other species such as rat or mouse, preferably a human-derived immunoglobulin Fc domain. The corresponding amino acid sequences of the rat immunoglobulin IgG1 Fc portion, mouse immunoglobulin IgG1 Fc portion and human immunoglobulin IgG1 Fc portion in the CFH-Ig fusion protein of the present invention are shown in SEQ ID NO:6, SEQ ID NO:7 and SEQ ID NO:8 of the Sequence Listing, respectively, or those amino acid sequences having at least 90% homology with SEQ ID NO:6, SEQ ID NO:7 and SEQ ID NO:8, respectively.

The order of arrangement of the CFH portion and the Fc portion of the CFH-Ig fusion protein of the present invention may be that the Fc portion is at the N-terminus and the CFH portion is at the C-terminus, i.e., Fc-CFH; or the CFH portion is at the N-terminus, and the Fc portion is at the C-terminus, i.e., CFH-Fc. In some embodiments, the CFH portion and the Fc portion are covalently linked; the covalent linker may be a peptide linker, such as (Gly4Ser)n, n should satisfy the maximum degree of assurance that the CFH and Fc portions are correctly assembled to exert its complement regulating functions; preferably, n is between 1 and 6. The covalent linker can also be a peptide bond connecting the CFH part and the Fc part. The covalent linker can further be any covalent connection that satisfies the maximum degree of assurance that the CFH and Fc portions are correctly assembled to achieve its complement regulating functions (e.g., chemical crosslinker). In the experiments of the present invention, the CFH-Ig fusion protein is directly linked by peptide bonds between the CFH portion and the Fc portion. In some embodiments, the CFH portion and the Fc portion may be non-covalently linked, e.g., the two portions may be linked through two interacting bridging proteins (e.g., biotin and streptavidin, or leucine zipper), with each bridging protein attached to either the CFH portion or the Fc portion.

In the present invention, the CFH-Ig fusion protein is linked by human SCR (1-7) and human Fc, and the sequence from the N-terminus to the C-terminus is hFc-L-hSCR (1-7) or hSCR (1-7)-L-hFc, where “h” represents human and “L” represents a peptide linker; for example, the sequence from the N-terminus to the C-terminus is hSCR (1-7)-L-hFc. In other embodiments, the CFH-Ig fusion protein is linked by human SCR (1-7) and mouse Fc, and the sequence from the N-terminus to the C-terminus is mFc-L-hSCR (1-7) or hSCR (1-7)-L-mFc, where “m” represents a mouse and “L” represents a peptide linker; for example, the sequence from the N-terminus to the C-terminus is hSCR(1-7)-L-mFc. In further embodiments, the recombinant CFH-Ig fusion protein is linked by human SCR (1-7), human SCR (18-20) and human Fc, and the sequence from the N-terminus to the C-terminus is hFc-L-hSCR (1-7)-hSCR (18-20) or hFc-L-hSCR (18-20)-hSCR (1-7) or hSCR (1-7)-hSCR (18-20)-L-hFc or hSCR (18-20)-hSCR (1-7)-L-hFc; for example, the sequence from the N-terminus to the C-terminus is hSCR (1-7)-hSCR (18-20)-L-hFc. In further embodiments, the CFH-Ig fusion protein is linked by human SCR (1-7), human SCR (18-20) and mouse Fc, and the sequence from the N-terminus to the C-terminus is mFc-L-hSCR (1-7)-hSCR (18-20) or mFc-L-hSCR (18-20)-hSCR (1-7) or hSCR (1-7)-hSCR (18-20)-L-mFc or hSCR (18-20)-hSCR (1-7)-L-mFc; for example, the sequence from the N-terminus to the C-terminus is hSCR (1-7)-hSCR (18-20)-L-mFc.

The peptide linker represented by “L” above may be (Gly4Ser)n, and “n” should satisfy the maximum degree of assurance for the correct assembly of the CFH portion and the Fc portion to achieve their complement regulating functions. Preferably, “n” is 0 or between 1 and 6; when “n” is 0, it means that the two parts of the fusion protein are linked by peptide bonds and not linked by the peptide “L”. Therefore, in the embodiments, the fusion protein corresponding to the above nomenclature all omits “L”, e.g., hSCR (1-7)-hFc, hSCR (1-7)-mFc and hSCR (1-7)-hSCR (18-20)-hFc, whose amino acid sequences are SEQ ID NO:9, SEQ ID NO:10 and SEQ ID NO:11 in the Sequence Listing, respectively, or those amino acid sequences having at least 90% homology with SEQ ID NO:9, SEQ ID NO:10, and SEQ ID NO:11 respectively.

In the present invention, the expression “SCR (1-3)” or “SCR 1-3” taken as an example means “fragment of SCR1 to SCR3”. The other numbers in the present invention have the same meaning.

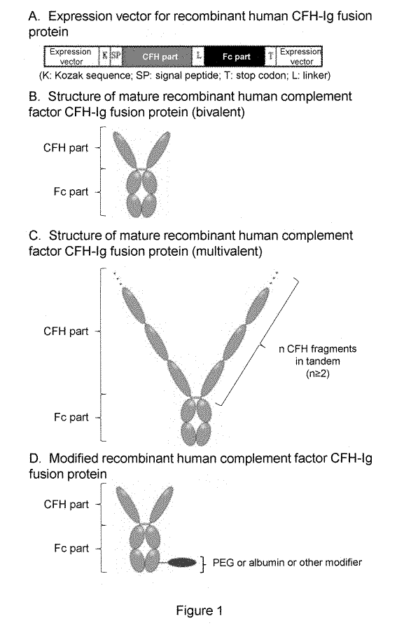

The term “valency” as used in the present invention refers to the specific number of CFH fragment contained in a single fusion protein, for example the term “bivalent” means that there are two CFH or two CFH fragments in a single fusion protein. The CFH-Ig fusion protein of the present invention is at least “bivalent”, and the structure of the mature recombinant human complement factor CFH-Ig fusion protein (bivalent) is shown in FIG. 1B, and may also be “multivalent” (e.g., “trivalent”, “tetravalent”, etc.). The above-stated “bivalent” is achieved by disulfide bond pairing between the two Fc fragments, resulting in a symmetric, antibody-like fusion protein. In some embodiments, the CFH portion in the CFH-Ig fusion protein may be linked to each other in tandem by two or more CFH fragments (same or different) (e.g., by fusion, or non-covalent linkage via a bridge protein), so as to form “multivalent” fusion protein; the structure of mature (multivalent) recombinant human complement factor CFH-Ig fusion protein is shown in FIG. 1C.

The CFH-Ig fusion proteins of the present invention also include, but are not limited to, the variants described below: (i) One or more amino acids of the CFH portion and/or the Fc portion of the immunoglobulin are substituted by conservative or non-conservative amino acids (preferably conservative amino acids) on the premise of maintaining complement regulatory activity, and the substituted amino acids may be those encoded by the genetic code, or those not encoded by the genetic code, or chemically synthesized non-natural amino acids; or (ii) Other amino acid sequences are fused to the fusion protein in the present invention to facilitate purification (e.g., His-tag, GST-tagged protein, etc.), or to facilitate secretory expression (e.g., signal peptide sequence), or to facilitate targeting to specific tissue or site such as complement receptor 2 (CR2) or complement receptor of the immunoglobulin superfamily (CRIg), or to increase half-life such as serum albumin; or (iii) Chemically modified variants including but not limited to polyethylene glycol (PEG) modifications, biotin modifications, and sugar chain modifications; modified recombinant human complement factor CFH-Ig fusion protein is schematically shown in FIG. 1D.

Genes encoding the above-described recombinant complement Factor H (CFH)-immunoglobulin (Ig) fusion protein (CFH-Ig) having a complement regulatory activity, in particular a complement regulatory activity in the alternative complement pathway, also fall within the scope of the present invention.

The expression vectors, transgenic cell lines and host cells containing the genes encoding the recombinant complement Factor H (CFH)-immunoglobulin (Ig) fusion protein (CFH-Ig) of the present invention also fall within the scope of the present invention.

Another object of the present invention is to provide a method for preparing a recombinant complement Factor H (CFH)-immunoglobulin (Ig) fusion protein (CFH-Ig).

Specifically, the method for preparing the CFH-Ig fusion protein of the present invention includes the following steps:

Step 1.

Synthesis of a cDNA sequence encoding a CFH-Ig fusion protein;

Step 2.

The cDNA sequence encoding the CFH-Ig fusion protein is inserted into a carrier vector to construct a recombinant expression vector that can be expressed in a host cell. The schematic structure thereof is shown in FIG. 1A;

Step 3.

The recombinant expression vector is transfected into a host cell for expression in a host cell;

Step 4.

The expressed CFH-Ig fusion protein is isolated and purified.

In the above preparation method of the CFH-Ig fusion protein, the carrier vector in the step 2 may be a commercially available carrier or a self-constructed expression carrier; the host cells include E. coli, yeast cells, and mammal cells, plant cells or insect cells. In one embodiment, the mammalian cell is a CHO cell.

The use as an active ingredient of a pharmaceutical of the recombinant complement Factor H (CFH)-immunoglobulin (Ig) fusion protein (CFH-Ig) having a complement regulatory activity, especially a complement regulatory activity in the alternative complement pathway, also fall within the scope of the present invention.

Provided in this invention is also a pharmaceutical composition that contains the aforementioned CFH-Ig fusion protein as a component of this composition.

The CFH-Ig fusion protein with various structures in the present invention is prepared into a pharmaceutical composition via pharmaceutically acceptable carriers suitable for administration. Suitable pharmaceutical carriers are well known to those skilled in the art and include, but are not limited to, physiological saline, phosphate buffer, water, liposomes, nano-carriers, etc. Pharmaceutical carriers containing CFH-Ig fusion proteins can be prepared by conventional methods.

The pharmaceutical composition of the present invention containing CFH-Ig fusion proteins of various structural types can be administered to humans or other mammals in effective dosages by various routes of administration, including but not limited to intravenous (iv), intravenous infusion, intramuscular injection (im), subcutaneous injection (sc), intravitreal injection (IVT), subconjunctival injection (SCJ), transscleral injection (TS), administration by intravitreal implantable devices, oral (po), sublingual administration (sl), spray, and eye drop. For different diseases, different routes of administration can be chosen. In some embodiments, the CFH-Ig fusion protein may be administered intravitreally (IVT), subconjunctival injection (SCJ), transscleral injection (TS), intravitreal implanted device, or eye drop; in other embodiments, the CFH-Ig fusion protein can be administered intravenously (iv), intravenous fusion, intramuscularly (im) or subcutaneously (sc).

The pharmaceutical composition of the present invention containing various types of recombinant CFH-Ig fusion proteins can be used alone or in combination with other drugs by the above administration route, or after being conjugated to other drugs for use in humans or other mammals. The other drugs include anti-vascular endothelial growth factor-A (VEGF-A) antibodies or antibody fragments, recombinant VEGF receptor fusion proteins or anti-C5 antibodies.

The pharmaceutical composition of the present invention containing recombinant CFH-Ig fusion proteins of various structural types can be used to prepare pharmaceuticals for the treatment of autoimmune diseases or other diseases mediated by alternative complement pathway or caused by disregulation or deficiency of the alternative complement pathway. The diseases include age-related macular degeneration (AMD), paroxysmal nocturnal hemoglobinuria (PNH), atypical hemolytic uremic syndrome (aHUS), type II membrane proliferative glomerulonephritis (Membranoproliferative glomerulonephritis type II). MPGN-II), or dense deposit disease (DDD).

The pharmaceutical composition of the present invention containing recombinant CFH-Ig fusion proteins of various structural types may also be coated on the surface of medical devices, particularly implantable medical devices such as artificial organs and cardiac stents, in direct contact with tissues or body fluids, or on the surface of extracorporeal shunting system, to inhibit thrombus formation caused by excessive activation of complement on the surfaces of these devices.

The CFH-Ig fusion protein with a recombinant complement Factor H (CFH) part and an immunoglobulin (Ig) part containing an immunoglobulin heavy chain constant region (CH) provided by the present invention, on the one hand, inhibits the overactivation of the alternative complement pathway through the action of the CFH part, including: (1) C3b binding activity; (2) cofactor activity as the cofactor of complement Factor I to cleave C3b; (3) C3bBb decay accelerating activity, and (4) activity inhibiting alternative complement pathway. In addition to complement modulating activity, the CFH-Ig fusion protein, particularly a fusion protein containing a C-terminal SCR (18-20) or SCR (19-20) fragment of the CFH molecule, also has the ability to simultaneously target the fusion protein to tissues or cells where excessive complement activation occurs. The CFH-Ig fusion protein of the present invention can be used to alleviate or treat related diseases mediated by the alternative complement pathway, or caused by disregulation or deficiency of the alternative complement pathway such as autoimmune diseases or other diseases, especially AMD, PNH, aHUS and MPGN-II. On the other hand, the half-life in vivo is prolonged by the effect of the constant region of the immunoglobulin heavy chain Fc to reduce the number of administrations and increase the compliance of the patient. The present invention has potential applications in the treatment of autoimmune diseases or other diseases mediated by the alternative complement pathway, or caused by disregulation or deficiency of the alternative complement pathway in humans or other mammals, and in the prevention or treatment of thrombus formation due to overactivation of complement system.

The following further describes the present invention in detail with reference to specific examples.

The methods used in the following Examples are all conventional methods unless otherwise specified.

Unless otherwise specified, the percent concentration is a percentage of mass/mass (w/w, unit g/100 g), mass/volume (w/v, unit g/100 mL), or volume/volume (v/v, unit mL/100 mL).

The way of obtaining the various materials described in the examples is merely to provide an example of a way of obtaining such materials for the purpose of this specific disclosure, and should not be taken as the limitation on the source of such materials in the present invention. In fact, the source of such materials used can be broad, and any such material that can be obtained through ways that do not violate the law and ethics can be replaced according to what is hinted in the examples.

The gene sequences used in the present invention were synthesized by Genscript (Nanjing) Co., Ltd.

The embodiments are implemented under the premise of the technical plans of the present invention, and give the detailed implementation way and the specific operation procedures. The embodiments are helpful for understanding the present invention, but the scope of protection of the present invention is not limited to the following embodiments.

### Example 1

### Design and Preparation of hSCR (1-7)-hFc Fusion Protein

1. Design and Synthesis of Nucleic Acid Sequence of SCR (1-7)-hFc Fusion Protein

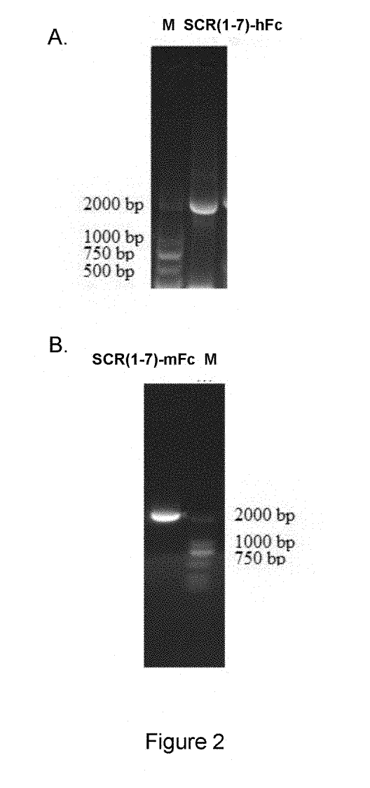

The amino acid sequences of human CFH (SEQ ID NO: AAI42700.1) and human IgG1 heavy chain (SEQ ID NO: CAA75032.1) were obtained from GenBank, and the sequences of SCR (1-7) from human CFH and Fc domain from human IgG1 were taken and linked by peptide bond from the N-terminus to the C-terminus. In order to facilitate the purification, a TEV (Tobacco etch virus protease) restriction site (ENLYFQG) and a 6×His tag were introduced at the C-terminus to obtain the molecule A1. The molecule A1 was then fused to the 3′ end of the human CFH signal peptide to give the molecule B1 (fusion protein human CFH signal peptide-hSCR (1-7)-hFc-6×His, where “h” is the first letter of the word “human”, representing human origin). Restriction sites Xho I and Xba I were introduced at the 5′ and 3′ ends of the coding sequence of molecule B1 to obtain the molecule C1 (fusion gene human Xho I-CFH signal peptide-hSCR (1-7)-hFc-6×His-Xba I). The gene sequence of the molecule C1 was codon optimized by Genscript (Nanjing) Co., Ltd. to obtain a nucleotide sequence that can be easily expressed in CHO cells and synthesized. This sequence is represented by SEQ ID NO:12 in the Sequence Listing. The 1% agarose gel electrophoresis result of the synthesized gene sequence is shown in FIG. 2A, and a band with 2079 bp gene was obtained, which was in agreement with the expected result.

2. Construction and Transfection of SCR (1-7)-hFc Fusion Protein Expression Vector and Screening of Stable Expression Cells

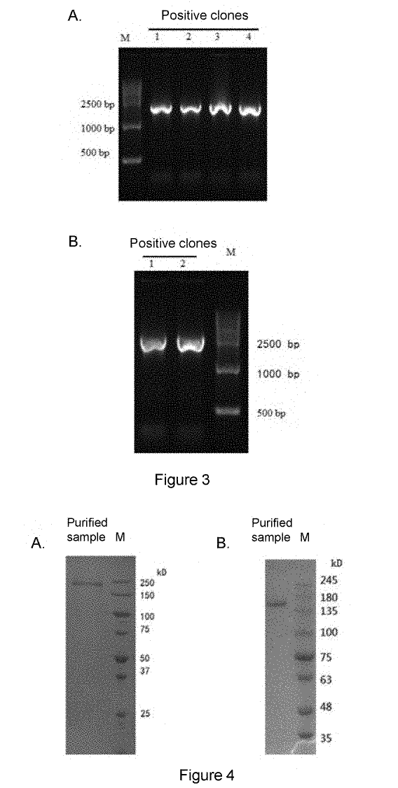

The fusion gene human Xho I-CFH signal peptide-hSCR (1-7)-hFc-6×His-Xba I whose sequence was proved to be correct by sequencing was digested by Xho I and Xba I enzymes into the pCI-neo vector (purchased from Promega). The positive clones were screened by PCR and the results are shown in FIG. 3A. Three positive clones were analyzed by DNA sequencing and were found to be identical to the designed sequences. CHO-DG44 adherent cells (purchased from Invitrogen) were then transfected with the positive clones, pressurized with 0.5 mg/mL G418, and stable expressing cells were isolated by limiting dilution methods. A stable cell line with high expression level (yield) was screened by Western method and the yield was 5 mg/L-100 mg/L.

3. Isolation and Purification of hSCR (1-7)-hFc Fusion Protein

The culture supernatant was collected and separated by Ni-NTA chelate chromatography and Protein A affinity chromatography in this order. Specifically, the supernatant of the culture medium was centrifuged at 1000 g for 10 minutes. The resulting supernatant was then loaded onto a Ni-NTA affinity column (purchased from GE Healthcare) pre-equilibrated with Solution I (20 mM Tris Cl+150 mM NaCl, pH 8.0), washed with 5-10 column volumes of Solution I. Then, impurities were eluted with solution II (20 mM TrisCl+150 mM NaCl+30 mM imidazole, pH 8.0). Then elute with solution III (20 mM TrisCl+150 mM NaCl+300 mM imidazole, pH 8.0) to collect the elution peak. The eluted materials were loaded onto a Protein A column (purchased from Millipore) pretreated with solution IV (PBS, 135 mM NaCl, 1.5 mM KH₂PO₄, and 8 mM K₂HPO₄, pH 7.4) and washed with solution IV for 5-10 column volume, and was further eluted with solution V (0.1 M glycine, pH 3.0) and the target elution peak was collected. Quantification of protein was performed using the BCA method. The isolated and purified expression product was detected by 8% SDS-PAGE. As shown in FIG. 4A, a 151 KD protein band was obtained, which was in agreement with the expected result, and the purity was >95%. The expressed hSCR (1-7)-hFc fusion protein was sequenced and its amino acid sequence was shown in SEQ ID NO:9 of the Sequence Listing.

4. Western Blot Identification of hSCR (1-7)-hFc Fusion Protein

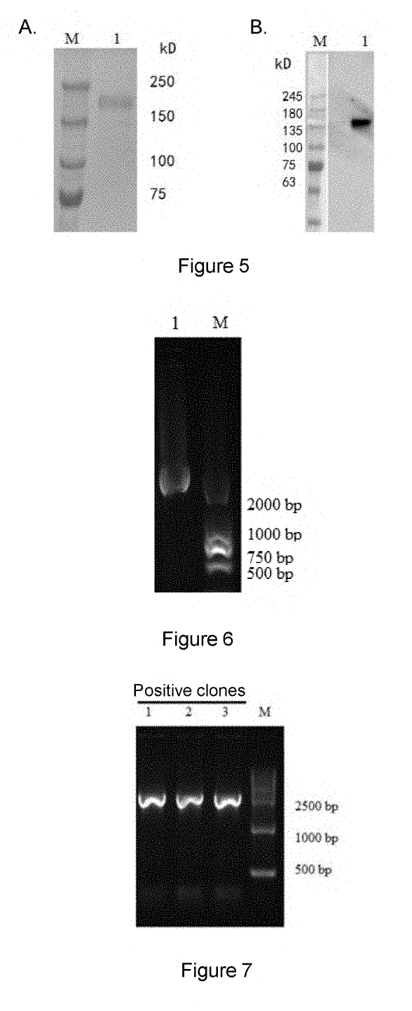

Protein samples purified from step 3 above were electrophoresed by 8% SDS-PAGE, then transferred to a PVDF membrane by an electrical transformer (Bio-Rad), and washed three times with TBS (50 mM Tris-C1, 150 mM NaCl, pH 7.5). After blocking for 1 h with 5% skimmed milk, they were incubated with mouse anti-human CFH monoclonal antibody (purchased from Santa Cruz) and goat anti-mouse monoclonal antibody-HRP (purchased from Biyuntian) for 2 h at room temperature. The color development was obtained with TMB (purchased from Biyuntian) after being washed three times with TBS. The results of the Western Blot assay are shown in FIG. 5A. It can be seen that there is a single band that is positive, indicating that the protein obtained is hSCR (1-7)-hFc.

### Example 2

### Design and Preparation of hSCR (1-7)-mFc Fusion Protein

1. Design and Synthesis of Nucleic Acid Sequence of SCR (1-7)-mFc Fusion Protein

The amino acid sequences of human CFH (SEQ ID NO: AAI42700.1) and mouse IgG1 heavy chain (SEQ ID NO: AAC08348.1) were obtained from GenBank and the Fc (1-7) and the sequences of SCR (1-7) from human CFH and Fc domain from mouse IgG1 were taken and linked by peptide bond from the N-terminus to the C-terminus. To facilitate the purification, a TEV restriction site and the 6×His tag were introduced at the C-terminus to obtain the molecule A2. The molecule A2 was then fused to the 3′ end of the human CFH signal peptide to give the molecule B2 (fusion protein human CFH signal peptide-hSCR (1-7)-mFc-6×His, where “h” and “m” are the first letters of “human” and “mouse”, respectively, representing human and mouse origin, respectively). Restriction sites Xho I and Xba I were introduced at the 5′ and 3′ ends of the coding sequence of molecule B2 to obtain the molecule C2 (fusion gene human Xho I-CFH signal peptide-hSCR (1-7)-mFc-6×His-Xba I). The gene sequence of the molecule C2 was codon optimized by Genscript (Nanjing) Co., Ltd. to obtain a nucleotide sequence that can be easily expressed in CHO cells and synthesized. This sequence is represented by SEQ ID NO:13 in the Sequence Listing. The 1% agarose gel electrophoresis result of the synthesized gene sequence is shown in FIG. 2B, and a band with 2064 bp gene was obtained, which was in agreement with the expected result.

2. Construction and Transfection of SCR (1-7)-mFc Fusion Protein Expression Vector and Screening of Stable Expression Cells

The fusion gene human Xho I-CFH signal peptide-hSCR (1-7)-mFc-6×His-Xba I whose sequence was proved to be correct by sequencing was digested by Xho I and Xba I enzymes into the pCI-neo vector (purchased from Promega). The positive clones were screened by PCR and the results are shown in FIG. 3B. Two positive clones were analyzed by DNA sequencing and were found to be identical to the designed sequences. CHO-DG44 adherent cells (purchased from Invitrogen) were then transfected with the positive clones, pressurized with 0.5 mg/mL G418, and stable expressing cells were isolated by limiting dilution methods. A stable cell line with high expression level (yield) was screened by Western method and the yield was 5 mg/L-100 mg/L. The expressed hSCR (1-7)-mFc fusion protein was sequenced and its amino acid sequence was shown in SEQ ID NO:10 in the Sequence Listing.

3. Isolation and Purification of hSCR (1-7)-mFc Fusion Protein

The culture supernatant was collected and separated by Ni-NTA chelate chromatography and Protein A affinity chromatography in this order. Specifically, the supernatant of the culture medium was centrifuged at 1000 g for 10 minutes. The resulting supernatant was then loaded onto a Ni-NTA affinity column (purchased from GE Healthcare) pre-equilibrated with Solution I (20 mM Tris Cl+150 mM NaCl, pH 8.0), washed with 5-10 column volumes of Solution I. Then, impurities were eluted with solution II (20 mM TrisCl+150 mM NaCl+30 mM imidazole, pH 8.0). Then elute with solution III (20 mM TrisCl+150 mM NaCl+300 mM imidazole, pH 8.0) to collect the elution peak. The eluted materials were loaded onto a Protein A column (purchased from Millipore) pretreated with solution IV (PBS, 135 mM NaCl, 1.5 mM KH₂PO₄, and 8 mM K₂HPO₄, pH 7.4) and washed with solution IV for 5-10 column volume, and was further eluted with solution V (0.1 M glycine, pH 3.0) and the target elution peak was collected. Quantification of protein was performed using the BCA method. The isolated and purified expression product was detected by 8% SDS-PAGE. As shown in FIG. 4B, a 151 KD protein band was obtained, which was in agreement with the expected result, and the purity was >95%.

4. Western Blot Identification of hSCR (1-7)-mFc Fusion Protein

Protein samples purified from step 3 above were electrophoresed by 8% SDS-PAGE, then transferred to a PVDF membrane by an electrical transformer (Bio-Rad), and washed three times with TBS (50 mM Tris-Cl, 150 mM NaCl, pH 7.5). After blocking for 1 h with 5% skimmed milk, they were incubated with mouse anti-human CFH monoclonal antibody (purchased from Santa Cruz) and goat anti-mouse monoclonal antibody-HRP (purchased from Biyuntian) for 2 h at room temperature. The color development was obtained with TMB (purchased from Biyuntian) after being washed three times with TBS. The results of the Western Blot assay are shown in FIG. 5B. It can be seen that there is a single band that is positive, indicating that the protein obtained is hSCR (1-7)-mFc.

### Example 3

### Design and Preparation of hSCR (1-7)-hSCR (18-20)-hFc Fusion Protein

1. Design and Synthesis of Nucleic Acid Sequence of hSCR (1-7)-hSCR (18-20)-hFc Fusion Protein

The amino acid sequences of human CFH (SEQ ID NO: AAI42700.1) and human IgG1 heavy chain (SEQ ID NO: CAA75032.1) were obtained from GeneBank, respectively, and the sequences of SCR (1-7) and SCR (18-20) from human CFH and Fc domain from human IgG1 were taken and linked from the N-terminus to the C-terminus by direct peptide bond between SCR (1-7) and SCR (18-20), and between SCR (18-20) and Fc. In order to facilitate the purification, a 6×His tag was introduced at the C-terminus to obtain the molecule A3. The molecule A3 was then fused to the 3′ end of the human CFH signal peptide to give the molecule B3 (fusion protein human CFH signal peptide-hSCR (1-7)-hSCR (18-20)-hFc-6×His, where “h” is the first letter of the word “human”, representing human origin). Restriction sites Xho I and Xba I were introduced at the 5′ and 3′ ends of the coding sequence of molecule B3 to obtain the molecule C3 (fusion gene human Xho I-CFH signal peptide-hSCR (1-7)-hSCR (18-20)-hFc-6×His-Xba I). The gene sequence of the molecule C3 was codon optimized by Genscript (Nanjing) Co., Ltd. to obtain a nucleotide sequence that can be easily expressed in CHO cells and synthesized. This sequence is represented by SEQ ID NO:14 in the Sequence Listing. The 1% agarose gel electrophoresis result of the synthesized gene sequence is shown in FIG. 6, and a band with 2640 bp gene was obtained, which was in agreement with the expected result.

2. Construction and Transfection of hSCR(1-7)-hSCR(18-20)-hFc Fusion Protein Expression Vector and Screening of Stable Expression Cells

The fusion gene human Xho I-CFH signal peptide-hSCR (1-7)-hSCR (18-20)-hFc-6×His-Xba I who sequence was proved to be correct by sequencing was digested by Xho I and Xba I enzymes into the pCI-neo vector (purchased from Promega). The positive clones were screened by PCR and the results are shown in FIG. 7. Three positive clones were analyzed by DNA sequencing and were found to be identical to the designed sequences. CHO-DG44 adherent cells (purchased from Invitrogen) were then transfected with the positive clones, pressurized with 0.5 mg/mL G418, and stable expressing cells were isolated by limiting dilution methods. A stable cell line with high expression level (yield) was screened by Western method and the yield was 5 mg/L-100 mg/L. The expressed hSCR (1-7)-hSCR (18-20)-hFc fusion protein was sequenced and its amino acid sequence was shown in SEQ ID NO:11 in the Sequence Listing.

3. Isolation and Purification of hSCR(1-7)-hSCR(18-20)-hFc Fusion Protein

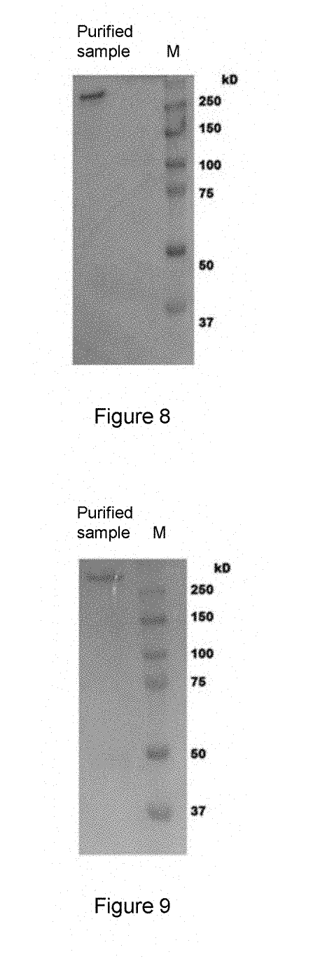

The culture supernatant was collected and separated by Ni-NTA chelate chromatography and Protein A affinity chromatography in this order. Specifically, the supernatant of the culture medium was centrifuged at 1000 g for 10 minutes. The resulting supernatant was then loaded onto a Ni-NTA affinity column (purchased from GE Healthcare) pre-equilibrated with Solution I (20 mM Tris Cl+150 mM NaCl, pH 8.0), washed with 5-10 column volumes of Solution I. Then, impurities were eluted with solution II (20 mM TrisCl+150 mM NaCl+30 mM imidazole, pH 8.0). Then elute with solution III (20 mM TrisCl+150 mM NaCl+300 mM imidazole, pH 8.0) to collect the elution peak. The eluted materials were loaded onto a Protein A column (purchased from Millipore) pretreated with solution IV (PBS, 135 mM NaCl, 1.5 mM KH₂PO₄, and 8 mM K₂HPO₄, pH 7.4) and washed with solution IV for 5-10 column volume, and was further eluted with solution V (0.1 M glycine, pH 3.0) and the target elution peak was collected. Quantification of protein was performed using the BCA method. The isolated and purified expression product was detected by 8% SDS-PAGE. As shown in FIG. 8, a 199 KD protein band was obtained, which was in agreement with the expected result, and the purity was >95%.

4. Western Blot Identification of hSCR(1-7)-hSCR(18-20)-hFc Fusion Protein

Protein samples purified from step 3 above were electrophoresed by 8% SDS-PAGE, then transferred to a PVDF membrane by an electrical transformer (Bio-Rad), and washed three times with TBS (50 mM Tris-C1, 150 mM NaCl, pH 7.5). After blocking for 1 h with 5% skimmed milk, they were incubated with mouse anti-human CFH monoclonal antibody (purchased from Santa Cruz) and goat anti-mouse monoclonal antibody-HRP (purchased from Biyuntian) for 2 h at room temperature. The color development was obtained with TMB (purchased from Biyuntian) after being washed three times with TBS. The results of the Western Blot assay are shown in FIG. 9. It can be seen that there is a single band that is positive, indicating that the protein obtained is hSCR (1-7)-hSCR (18-20)-hFc.

### Example 4

### Comparison of Binding Affinity to C3b of hSCR (1-7)-hFc, hSCR (1-7)-mFc, hSCR (1-7)-hSCR (18-20)-hFc and Human CFH

The binding affinity of hSCR (1-7)-hFc, hSCR (1-7)-mFc, hSCR (1-7)-hSCR (18-20)-hFc, and human CFH to C3b was detected by ELISA. The specific method was: 96 well plates were coated with 100 μL C3b at the final concentration of 5 μg/mL at 4° C. overnight, washed three times with PBST (PBS+0.1% Tween 20) the next day, blocked with 200 μL 5% skim milk for 2 h, and then washed three times with PBST. A series of concentrations (diluted from the initial 60 nM to 60 nM, 30 nM, 15 nM, 7.5 nM) of test samples (hSCR (1-7)-hFc, hSCR (1-7)-mFc and hSCR (1-7))-hSCR (18-20)-hFc) were added to the plates and incubated for 2 h, washed three times with PBST, incubated with 1:5000 primary antibody (mouse anti-human CFH antibody) for 2 h, washed three times with PBST, and then incubated with 1:5000 secondary antibody (HRP-conjugated goat anti-mouse monoclonal antibody) for 2 h, finally washed with PBST, color developed with TMB, and terminated with 2 M sulfuric acid. The absorbance at 450 nM wavelength was measured. Human CFH was used as a positive control, PBS as a negative control, and three replicates were used for each sample.

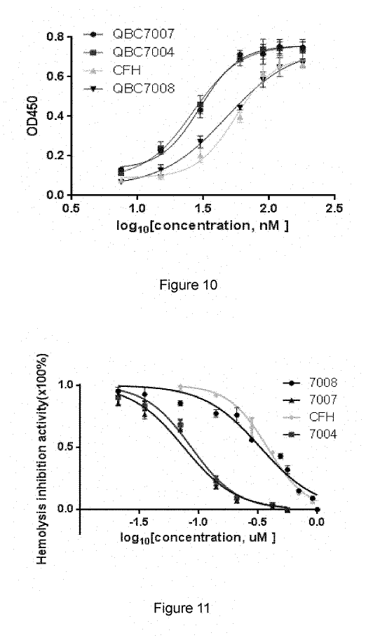

The results are shown in FIG. 10, where QBC7007 refers to hSCR (1-7)-hFc, QBC7004 refers to hSCR (1-7)-mFc, and QBC7008 refers to hSCR (1-7)-hSCR (18-20)-hFc. It can be seen that the C3b binding affinity of SCR (1-7)-hFc and SCR (1-7)-mFc is comparable and significantly higher than that of human CFH, while the binding affinity of SCR (1-7)-SCR (18-20)-hFc is close to that of human CFH.

### Example 5

### Comparison of Inhibitory Activity on Hemolysis of hSCR (1-7)-hFc, hSCR (1-7)-mFc, hSCR (1-7)-hSCR (18-20)-hFc and Human CFH

1. Materials and Preparation

Preparation of 5×VBS (500 mL): weighed 1.15 g of barbituric acid and dissolved in 200 mL of boiling water; weighed 1.15 g of sodium barbiturate and 20.95 g of NaCl and dissolved in 250 mL of water; added water to 500 mL after cooling. The final solution was adjusted to pH between 7.2-7.4 with NaOH.

0.1M MgEGTA (100 mL): weighed 3.80 g of EGTA (Sigma), 2.03 g of MgCl₂.6H₂O (Amersco) and added 90 mL of water. The final solution was adjusted to pH 7.2-7.4 with NaOH, and the volume was set to 100 mL.

GVB (200 mL, prepared fresh): Took 40 mL 5×VBS, 0.2 g gelatin (Fluka), dissolved in 150 mL ultrapure water, dissolved the gelatin completely in a 45° C. water bath, adjusted the pH to 7.2-7.4, set the volume to 200 mL, and filtered with 0.22 μm filter before use.

GVBE (100 mL, prepared fresh): Took 20 mL of 5×VBS, 0.1 g of gelatin (Fluka), and 0.37 g of EDTA-Na2 (Amresco), dissolved in 70 mL of ultrapure water, and completely dissolved the gelatin in a 45° C. water bath, adjusted the pH to 7.3, set the volume to 100 mL, and filtered with 0.22 μm.

Treatment and counting of rabbit erythrocytes: Took defibrinated rabbit blood, washed once with GVBE, washed twice with GVB, counted, and then diluted to 5×10<8>/mL. Took 254, added 1 mL of pure water to produce hemolysis, and measured absorption value at 412 nm to be about 1.3.

Preparation of NHS (½) (normal human serum): Diluted NHS half with GVB and kept on ice.

2. Determination of the Amount of NHS (½) that Produced 50% Hemolysis

Under normal conditions, sialic acid can inhibit the activity of Factor B. Rabbit erythrocytes contain less sialic acid than red blood cells in other animals and can activate B factor in the serum, causing activation of the alternative complement pathway, leading to lysis of rabbit red blood cells. At a fixed amount of red blood cells, under a given reaction condition, the degree of hemolysis is positively correlated with the amount of complement and its activity involved in alternative complement activation in the serum.

Various reagents were added to the 2 mL round-bottom tubes according to the order and concentration shown in Table 1. Each group was run in duplicate and the unit was μL. Tubes were added reagents sequentially in an ice bath, transferred to a 37° C. water bath, incubated for 30 minutes, and shaken once every 5 minutes. Add 1 mL of ice-cold GVBE, mix and centrifuge at 1000 g for 3 minutes. The supernatant was removed and the absorbance at 412 nm was measured. Among them, Tube 1 is a background sample, and its reading was subtracted from all other readings. Tube 2 is the result of 100% hemolysis. Tube 3 served as a blank control. The obtained absorption value of each tube was divided by the Tube 2 absorption value to get the percentage hemolysis. Curve fitting was performed using Graph Prism 6, and the volume of NHS (½) required for 50% hemolysis was 18 μL. The amount of 18 μL of NHS was thus used in the following hemolysis experiment since it was the most sensitive point to detect hemolysis in response to complement activity.

TABLE 1
Experimental protocol to determine the
amount of NHS (½) that produces 50% hemolysis

| | Tube No. | | | | | | | | | | |
|---|---|---|---|---|---|---|---|---|---|---|---|
| Reagents | 1 | 2 | 3 | 4 | 5 | 6 | 7 | 8 | 9 | 10 | 11 |
| GVB | — | — | 70 | 66 | 62 | 58 | 54 | 50 | 46 | 42 | 38 |
| (μL) | | | | | | | | | | | |
| GVBE | 55 | — | — | — | — | — | — | — | — | — | — |
| (μL) | | | | | | | | | | | |
| Water | — | 55 | — | — | — | — | — | — | — | — | — |
| (μL) | | | | | | | | | | | |
| 0.1M MgEGTA | — | — | 5 | 5 | 5 | 5 | 5 | 5 | 5 | 5 | 5 |
| (μL) | | | | | | | | | | | |
| NHS (½) | 20 | 20 | 0 | 4 | 8 | 12 | 16 | 20 | 24 | 28 | 32 |
| (μL) | | | | | | | | | | | |
| Er[5 × 10⁸/mL] | 25 | 25 | 25 | 25 | 25 | 25 | 25 | 25 | 25 | 25 | 25 |
| (μL) | | | | | | | | | | | |

3. Hemolysis Inhibition Experiment

CFH is an important negative regulator of the alternative complement pathway. CFH determines the fate of complement C3b, whether within the blood vessels or on the cell surface, and controls the formation of C3 convertase and its stability. Therefore, in the above experiment, the addition of CFH inhibits the hemolytic activity of the complement-mediated alternative pathway in serum. The experimental method for the inhibition of hemolysis of rabbit red blood cells is the same as the above experiment. The amount of NHS added was based on 50% of the amount required for hemolysis. 100 μL of the reaction system was added with different concentrations of the samples, and the absorbance was measured at a wavelength of 412 nm after 30 minutes of reaction. A comparison of the hemolytic inhibitory activity of hSCR (1-7)-hFc, hSCR (1-7)-mFc, hSCR (1-7)-hSCR (18-20)-hFc, and human CFH is shown in FIG. 11. IC₅₀ values could be calculated according to the curve fitting, as shown in Table 2, where 7007 refers to hSCR (1-7)-hFc, 7004 refers to hSCR (1-7)-mFc, and 7008 refers to hSCR (1-7)-hSCR (18-20)-hFc. According to this experimental result, the hSCR (1-7)-hFc had the highest activity in inhibiting the hemolysis, 5 times higher than that of human CFH, followed by hSCR (1-7)-mFc, which was also significantly higher than that of human CFH activity, and hSCR (1-7)-hSCR (18-20)-hFc, which was comparable to human CFH activity, indicating that the designed recombinant proteins all had the expected biological activity.

Table 2 Results of hemolytic inhibition activity (IC₅₀ values)

| Tested samples | IC₅₀ |
|---|---|
| 7007 | 0.074 μM |
| 7008 | 0.33 μM |
| 7004 | 0.086 μM |
| Human CFH | 0.37 μM |

### Example 6

### Comparison of Co-Factor Activity of hSCR (1-7)-hFc, hSCR (1-7)-mFc, hSCR (1-7)-hSCR (18-20)-hFc and Human CFH, for Factor I Cleavage of C3b

CFH can act as co-factor for Factor I to cleave the subunit of C3b (101 kD), forming bands of 68 kD and 43 kD size on the electropherogram. This experiment compared co-factor activities of hSCR (1-7)-hFc, hSCR (1-7)-mFc, hSCR (1-7)-hSCR (18-20)-hFc, and human CFH in assisting Factor I to cleave C3b. Test samples were added to the tube according to the experimental design as shown in Table 3 (in ice bath), mixed and incubated at 37° C. water bath. 10 μL was taken at 5 min and 30 min respectively, DTT was added to final concentration of 100 mM, stopped by adding electrophoresis loading buffer, and then subjected to 8% SDS-PAGE electrophoresis. Human CFH was used as a positive control and PBS as a negative control instead of the sample.

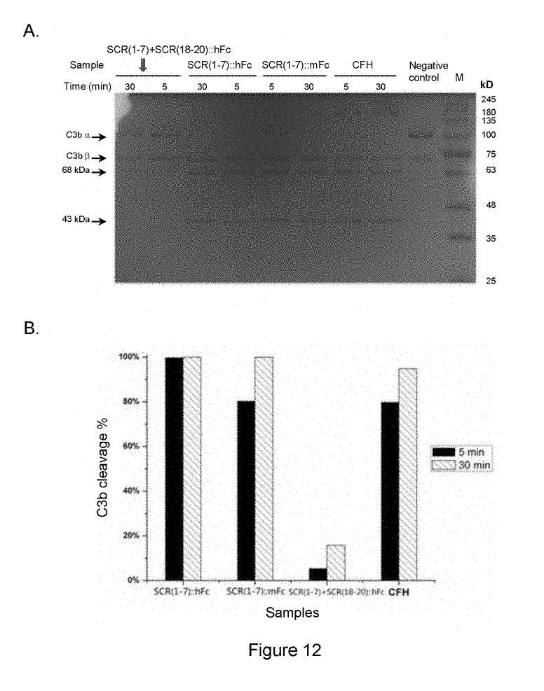

The result with 8% of SDS-PAGE electrophoresis is shown in FIG. 12A, and the cleavage rate of the C3bα subunit was analyzed by optical density analysis. The comparison result is shown in FIG. 12B, and it can be seen that the fusion proteins hSCR (1-7)-hFc and hSCR(1-7)-mFc did not have lower co-factor activities than control human CFH in assisting Factor I to cleave C3b, but hSCR (1-7)-hSCR (18-20)-hFc had lower activity than CFH in this experiment.

TABLE 3
Experimental protocol for comparing co-factor activity
for Factor I Cleavage of C3b Activity

| | Tube No | | | | |
|---|---|---|---|---|---|
| | 1 | 2 | | | |
| Ingredients | (negative) | (positive) | 3 | 4 | 5 |
| fI (0.2 mg/mL) | 1 μL | 1 μL | 1 μL | 1 μL | 1 μL |
| C3b (0.33 mg/mL) | 3 μL | 3 μL | 3 μL | 3 μL | 3 μL |
| CFH (1.0 mg/mL) | — | 1 μL | — | — | — |
| SCR (1-7)-mFc | — | — | 10 μL | — | — |
| (0.1 mg/mL) | | | | | |
| SCR (1-7)-hFc | — | — | — | 5 μL | — |
| (0.2 mg/mL) | | | | | |
| SCR (1-7)-SCR | — | — | — | — | 10 μL |
| (18-20)-hFc | | | | | |
| (0.1 mg/mL) | | | | | |
| PBS | 16 μL | 15 μL | 6 μL | 11 μL | 6 μL |
| Total | 20 μL | 20 μL | 20 μL | 20 μL | 20 μL |

Based on the results of Example 4, Example 5, and Example 6, it was concluded that hSCR (1-7)-hFc and hSCR (1-7)-mFc are the most preferred CFH-Ig fusion proteins of the present invention.

In summary, the present invention fuses a human CFH fragment with an immunoglobulin Fc fragment to form a new structure, and the preferred CFH-Ig fusion protein has a higher inhibitory effect on the alternative complement pathway compared to natural CFH. The fusion protein also contains an immunoglobulin heavy chain constant region Fc fragment, which can prolong its half-life in vivo so as to reduce the number of administrations and increase the patient's compliance. Therefore, the CFH-Ig fusion protein of the present invention can be used in the preparation of pharmaceutical composition for the treatment of various diseases mediated by alternative complement pathway, or caused by disregulation or deficiency in alternative pathway, such as autoimmune diseases (e.g., rheumatoid arthritis) or other diseases (e.g., ischemia and reperfusion), especially age-related macular degeneration (AMD), paroxysmal nocturnal hemoglobinuria (PNH), atypical hemolytic uremic syndrome (aHUS) and type II membranoproliferative glomerulonephritis (membranoproliferative glomerulonephritis type II, MPGN-II) or dense deposit disease (DDD); the CFH-Ig fusion protein of the present invention may also be applied to the surfaces of medical devices, especially implantable medical devices in direct contact with tissue or body fluids (including but not limited to blood) such as artificial organs, cardiac stents, pacemakers, implantable sensing-telemetry systems, and extracorporeal blood bypass systems to inhibit thrombus formation caused by excessive activation of complement on its surface.

In the prior art, although some patents have used CFH, full-length CFH, or different CFH fragments (such as SCR (1-5) in U.S. Pat. No. 7,759,304B2), the CFH portion in the CFH-Ig fusion proteins in the present invention contains fragment that regulates the alternative complement pathway, or fragment that targets the fusion proteins to tissues where overactivation of complement occurs. The SCR 7 in fragment SCR (1-7) and SCR (19-20) in fragment SCR (18-20) are binding domains of GAG and CRP, can effectively regulate complement activation by binding to GAG or CRP in tissues or cells where C3b is deposited on their surfaces due to overactivation of complement, and can thus target fragment SCR (1-4) with complement inhibitory activity to tissues or cell surfaces where excessive complement activation occurs. Furthermore, the CFH fusion protein disclosed in the present invention also contains an immunoglobulin heavy chain constant region Fc fragment, which can prolong its half-life in vivo, so as to reduce the number of administrations and increase the patient's compliance. Therefore, the present invention utilizes the multiple functions of complement Factor H to disclose a fusion protein that contains fragments that can modulate the complement system, particularly the alternative complement pathway, or simultaneously contains fragments that have the effect of targeting complement inhibitory part of the fusion protein to tissues or cell surfaces where excessive complement activation occurs. The CFH-Ig fusion protein disclosed in the present invention also contains an immunoglobulin heavy chain constant region Fc fragment, which can prolong its half-life in vivo.

The fusion protein CFH-Ig disclosed in the present invention has the complement regulatory activity, especially the complement regulatory activity in the alternative complement pathway, or simultaneously has the function of targeting to tissues with excessive complement activation. The fusion protein CFH-Ig can be used in the preparation of pharmaceutical composition for the treatment of related diseases.

What is claimed is:

1. A recombinant complement Factor H (CFH)-immunoglobulin (Ig) fusion protein, having a complement regulatory activity, comprising:
a) a complement Factor H part selected from the group consisting of a full-length CFH, a biologically active CFH fragment, and a combination of fragments thereof, and
b) an immunoglobulin part having an immunoglobulin heavy chain constant region (CH),
wherein the CFH part has a complement regulatory activity having an effect selected from the group consisting of inhibiting the excessive complement activation, regulating the excessive complement activation, and simultaneously with the aforementioned effects targeting to tissues where there is excessive complement activation; and
wherein the immunoglobulin part has an effect of prolonging its half-life in vivo.

2. The fusion protein according to claim 15, wherein the CFH part is selected from the group consisting of at least one full-length CFH sequence, at least one biologically active N-terminal short consensus repeat (SCR) fragment of CFH from SCR1-SCR17, a combination of at least two different aforementioned SCR fragments each with at least one aforementioned SCR fragment, a combination of at least one aforementioned SCR fragment with C-terminal SCR (18-20) of CFH, a combination of at least one aforementioned SCR fragment with C-terminal SCR (19-20) of CFH, a combination of two different combined aforementioned SCR fragments with C-terminal SCR (18-20) of CFH, and a combination of two different combined aforementioned SCR fragments with C-terminal SCR (19-20) of CFH, wherein the N-terminal SCR fragment of CFH is selected from the group consisting of SCR (1-3), SCR (1-4), SCR (1-5), SCR (1-6), SCR (1-7), SCR (1-8), SCR (1-9), SCR (1-10), SCR (1-11), SCR (1-12)), SCR (1-13), SCR (1-14), SCR (1-15), SCR (1-16) and SCR (1-17); the numbers for more than one aforementioned N-terminal SCR fragments in the fusion protein being in the range of 2 to 4.

3. The fusion protein according to claim 2, wherein the CFH part is selected from the group consisting of full-length CFH sequence, CFH fragment SCR (1-4), CFH fragment SCR (1-7), a combination of SCR (1-4) and SCR (18-20), a combination of SCR (1-4) and SCR (19-20), a combination of SCR (1-7) and SCR (18-20), and a combination of SCR (1-7) and SCR (19-20).

4. The fusion protein according to claim 3, wherein the human full-length CFH, human CFH fragment SCR (1-4), human CFH fragment SCR (1-7), human CFH fragment SCR (18-20) and human CFH fragment SCR (19-20) have the corresponding amino acid sequences as shown in SEQ ID NO:1, SEQ ID NO:2, SEQ ID NO:3, SEQ ID NO:4 and SEQ ID NO:5, respectively, in the Sequence Listing, or have amino acid sequences that have at least 90% homology with those as shown in SEQ ID NO:1, SEQ ID NO:2, SEQ ID NO:3, SEQ ID NO:4 and SEQ ID NO:5, respectively, in the Sequence Listing.

5. The fusion protein according to claim 4, wherein the CFH part is derived from humans, but also derived from other species selected from the group consisting of mice, rats, guinea pigs, rabbits, dogs, pigs, sheep, and non-human primates; preferably, wherein the CFH part is derived from the group consisting of humans, mice, rats, and non-human primates; more preferably, wherein the CFH part is derived from humans.

6. The fusion protein according to claim 16, wherein the immunoglobulin heavy chain constant region includes a CH1 region, a hinge region, a CH2 region, and a CH3 region, and the immunoglobulin heavy chain constant region including at least a Fc fragment (hinge region, CH2 region and CH3 region); the Fc portion being an immunoglobulin Fc domain derived from the group consisting of human, rat and mouse; preferably the Fc portion being from human; the corresponding amino acid sequences of the Fc portion of rat, mouse and human immunoglobulin IgG1 being shown in SEQ ID NO:6, SEQ ID NO:7 and SEQ ID NO:8, respectively, of the Sequence Listing, or amino acid sequences having at least 90% homology with those as shown in SEQ ID NO:6, SEQ ID NO:7 and SEQ ID NO:8, respectively.

7. The fusion protein according to claim 6, wherein the protein is a fusion protein of CFH and Fc, and the order of sequence of the two parts being a CFH part at the N-terminus and a Fc part at the C-terminus (CFH-Fc); the CFH and Fc parts being covalently linked, and the covalent link being a peptide linker (Gly4Ser)n, where n is 0 or between 1 and 6, and n being 0 means that CFH and Fc parts are directly linked by peptide bonds; the CFH and Fc parts also being non-covalently linked and being mediated by two interacting bridging proteins, each bridging protein being connected to the CFH part or Fc part.

8. The fusion protein according to claim 7, selected from the group consisting of the following:
fusion protein of human SCR (1-7) and human Fc, and the sequence from the N-terminus to the C-terminus being hSCR (1-7)-L-hFc, where “h” represents human and “L” represents a peptide linker;
fusion protein of human SCR (1-7) and mouse Fc, and the sequence from the N-terminus to the C-terminus being hSCR (1-7)-L-mFc, wherein “m” represents mouse and “L” represents a peptide linker;
fusion protein of human SCR (1-7), human SCR (18-20) fused to human Fc, wherein the sequence from the N-terminus to the C-terminus is hSCR (1-7)-hSCR (18-20)-L-hFc; and
fusion protein of human SCR (1-7), human SCR (18-20) fused to mouse Fc, wherein the sequence from N-terminus to the C-terminus is hSCR (1-7)-hSCR (18-20)-L-mFc; and
where n is 0, the amino acid sequences of the fusion proteins hSCR (1-7)-hFc, hSCR (1-7)-mFc, and hSCR (1-7)-hSCR (18-20)-hFc being shown in SEQ ID NO:9, SEQ ID NO:10, and SEQ ID NO:11, respectively, of the Sequence Listing, or amino acid sequences having at least 90% homology with those in SEQ ID NO:9, SEQ ID NO:10, and SEQ ID NO:11, respectively.

9. The fusion protein according to claim 8, wherein the CFH-Ig fusion protein is at least “bivalent” or “multivalent”, and the “bivalent” being achieved by disulfide bond pairing between the two Fc fragments, resulting in a symmetric, antibody-like fusion protein; the CFH parts being linked to each other in tandem by two or more CFH fragments, which are same or different, to form “multivalent” fusion proteins.

10. (canceled)

11. (canceled)

12. (canceled)

13. The fusion protein according to claim 1, which is prepared into a pharmaceutical composition for the treatment of human or other mammalian diseases; the disease being selected from the group consisting of age-related macular degeneration (AMD), paroxysmal nocturnal hemoglobinuria (PNH), atypical hemolytic uremic syndrome (aHUS), type II membrane proliferative glomerulonephritis (Membranoproliferative glomerulonephritis type II, MPGN-II), dense deposition disease (DDD), and for the prevention and treatment of a thrombus formation on the surface of medical device.

14. The fusion protein according to claim 13, wherein the medical device comprises an implantable medical device selected from the group consisting of artificial organs, heart stents, extracorporeal shunt systems that are in direct contact with tissue or body fluids.

15. The fusion protein according to claim 1, wherein the complement regulatory activity of the CFH part is an activity in the alternative complement pathway.

16. The fusion protein according to claim 5, wherein the immunoglobulin part is derived from the group consisting of humans, rats and mice; the immunoglobulin heavy chain constant region being selected from different immunoglobulins such as IgA, IgD, IgE, IgG and IgM, preferably IgG; the immunoglobulin G (IgG) being selected from different subtypes of IgG, namely IgG1, IgG2 (IgG2a, IgG2b), IgG3 and IgG4, and combinations between different subtypes (e.g., IgG2/IgG4); more preferably, the immunoglobulin heavy chain constant region being selected from IgG1, IgG2 and IgG4.

17. A method of preparing a recombinant complement Factor H (CFH)-immunoglobulin (Ig) fusion protein according to claim 1 having complement regulatory activity, comprising:
synthesizing a cDNA sequence encoding a CFH-Ig fusion protein;
inserting the cDNA sequence encoding the CFH-Ig fusion protein into a carrier vector to construct a recombinant expression vector that can be expressed in a host cell;
transfecting the recombinant expression vector into a host cell for expression in the host cell thus obtaining an expressed CFH-Ig fusion protein;
isolating and purifying the expressed CFH-Ig fusion protein.

18. The method according to claim 17, wherein the carrier vector is commercially available carrier or a self-constructed expression carrier and wherein the host cells include E. coli, yeast cells, and mammal cells, plant cells or insect cells.

19. The method according to claim 18, wherein the mammal cell is a CHO cell.

20. A composition comprising a recombinant complement Factor H (CFH)-immunoglobulin (Ig) fusion protein which is an active substance having a complement regulatory activity and comprising:
a) a complement Factor H part selected from the group consisting of a full-length CFH, a biologically active CFH fragment, and a combination of fragments thereof, and
b) an immunoglobulin part having an immunoglobulin heavy chain constant region (CH),
wherein the CFH part has a complement regulatory activity having an effect selected from the group consisting of inhibiting the excessive complement activation, regulating the excessive complement activation, and simultaneously with the aforementioned effects targeting to tissues where there is excessive complement activation; and wherein the immunoglobulin part has an effect of prolonging its half-life in vivo.


SEQUENCE LISTING (14 sequences)

SEQ ID NO: 1 - protein, 1213 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 2 - protein, 246 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEE

SEQ ID NO: 3 - protein, 424 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCI

SEQ ID NO: 4 - protein, 187 aa, Homo sapiens
DTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 5 - protein, 127 aa, Homo sapiens
DSGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 6 - protein, 229 aa, Rattus
VPRNCGGDCKPCICTGSEVSSVFIFPPKTKDVLTITLTPKVTCVVVDISQNDPEVRFSWFIDDVEVHTAQTHAPEKQSNSTLRSVSELPIVHRDWLNGKTFKCKVNSGAFPAPIEKSISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 7 - protein, 227 aa, Mus
VPRDCGCKPCICTVPEVSSVFIFPPKPKDVLTITLTPKVTCVVVDISKDDPEVQFSWFVDDVEVHTAQTQPREEQFASTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 8 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 656 aa, Artificial sequence
  Fusion protein hSCR(1-7)-hFc
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 652 aa, Artificial sequence
  Fusion protein hSCR(1-7)-mFc
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIVPRDCGCKPCICTVPEVSSVFIFPPKPKDVLTITLTPKVTCVVVDISKDDPEVQFSWFVDDVEVHTAQTQPREEQFASTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 11 - protein, 611 aa, Artificial sequence
  Fusion protein hSCR(1-7)-hSCR(18-20)-hFc
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 12 - DNA, 2079 bp, Artificial sequence
  Fusion gene human Xho I-CFH signal peptide-hSCR(1-7)-hFc-6XHis-Xba I
ctcgagatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagagaacctgtacttccagggacaccaccaccaccaccactagtctaga

SEQ ID NO: 13 - DNA, 2064 bp, Artificial sequence
  Fusion gene human Xho I-CFH signal peptide-hSCR(1-7)-mFc-6XHis-Xba I
ctcgagatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatcgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcgctagcactttccgctcagtcagtgaacttcccatcatgcaccaggactggctcaatggcaaggagttcaaatgcagggtaaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaagagaacctgtacttccagggacaccaccaccaccaccactagtctaga

SEQ ID NO: 14 - DNA, 2640 bp, Artificial sequence
  Fusion gene human Xho I-CFH signal peptide-hSCR(1-7)-hSCR(18-20)-hFc-6XHis-Xba I
ctcgagatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatcgacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagagaacctgtacttccagggacaccaccaccaccaccactagtctaga